(19) United States
(12) Patent Application Publication
(10) Pub. No.: US 2011/0136776 A1
(43) Pub. Date: Jun. 9, 2011

(54) Copper(1)Chloride Complex of Nicotinic Acid and Pharmaceutical Compositions Containing the Same

(76) Inventor: Salah Fathiy Hussein Aly Ibraheim, Cairo (EG)

(21) Appl. No.: 13/055,428

(22) PCT Filed: Sep. 21, 2008
(86) PCT No.: PCT/EG2008/000034

§ 371 Date: Jan. 21, 2011

(30) Foreign Application Priority Data
Jul. 22, 2008 (EG) 2008071244

(51) Int. Cl. A61K 31/555; C07F 1/08; A61P 21/00; A61P 17/02; A61P 21/04; A61P 25/16; A61P 15/08

(52) U.S. Cl. 514/188; 546/5

(57) ABSTRACT

Copper (I) chloride complex of nicotinic acid and pharmaceutical compositions containing the same. Such pharmaceutical composition are effective for the treatment of myopathy, myasthenia gravis, parkinsonism, Chronic Fatigue Syndrome, Male Infertility and post stroke muscle weakness, muscular dystrophy, skin burns, scars, alopecia, high blood lipids and obesity.

### TECHNICAL FIELD

Copper (I) chloride complex of nicotinic acid and pharmaceutical compositions containing the same.

### BACKGROUND ART

Copper (I) chloride complex of nicotinic acid is prepared, characterized by elemental analysis, IR, UV-visible spectra and its crystal structure determined by single crystal diffraction methods. This compound is found to exhibit a very positive influence as a drug in a pharmaceutical acceptable composition for different incurable diseases, e.g. rnyopathy or weakness of muscles in general, infertility, etc.

Duchene dystrophy (DD) is an x linked disorder primarily affecting skeletal muscle, caused by lack of dystrophin—the protein product of DD gene located on XP21. These patients are males who suffer from progressive muscle weakness extending to both cardiac and respiratory muscle failure. DD patients die at the age of 25-30 years old of either respiratory and/or cardiac failures. Females are healthy carriers. Several trials for both drug and genetic treatment did not give satisfactory results.

Male infertility is a multi factorial disease process with a number of potential contributing causes. Considering the majority of male infertility cases are due to deficient sperm production of unknown origin, environmental and mutational factors must be evaluated.

The treatment of male factor infertility is a rapidly developing field. Introduction of microsurgical fertilization techniques allows assisted conception units to treat couples who previously would not have benefited from in vitro fertilization techniques. However these techniques are only used for the minority of sub-fertile men in andrological practice. Many subfertile men are still treated pharmacologically or by sperm selection methods to enhance sperm fertilization ability. Numerous pharmacological compound have been described that enhance sperm motility and thus potentially sperm fertilizing capacity. Sperm motility plays an important role in the normal fertilization process. Poor sperm motility (<50% motile sperm with <2+ forward progression according to WHO protocol) is considered a major factor in diminished rates of fertilization Medical trials for male non obstructive infertility by hormonal replacement, corticosteroids (in immunes infertility), mutational therapies but results were not satisfactory. Chronic fatigue is world wide complaint affecting the productivity of a good percentage of the population. Chronic fatigue includes unexplained fatigue, chronic fatigue syndrome and fibromyalgia. In all types of fatigue the complex relieved the muscle pain, increased the physical activity and productivity and associated depression.

### RELATIONS TO PREVIOUS ARTS IN THE SAME FIELD

The new art provides a modified method for preparation to overcome the recorded defects in the previous art.
The present application is based on a previous registered PCT EGY 116/030/2006
Copper Nicotinate, The active ingredient in (Royal Top)® preparations is dispensed in soft gelatin capsule together with Royal Jelly and Vitamin E.
To our knowledge, there's no available literature about the methods of analysis of copper (I) in the complex Copper Nicotinate or analysis of the complex in the formulation.
Developments of selective and reliable methods for analyses are mandatory for stability study of the formulation and pharmacokinetic determinations.
The above mentioned parameters are prerequisites for launching pharmaceutical dosage forms which is not at hand till present.
The formulation procedure contains water which is contraindicated with the stability of copper (I).
The Iron oxide added to the formula affords probability of displacement of copper from the active ingredient (copper Nicotinate) or the interference with the balance of copper and other elements in the biological system.
The Parabenzoate added as preservative, the pharmacological undesirable effect is questionable as recorded in recent literature.
Among the observed defects there's no available reference about the probable drug interaction with other drugs e.g. salicylates, phenobarbital and NSAIDs.
Preparation of the copper nicotinate complex according to the procedure of the previous claim didn't give a reproducible results cooping with the declared constants.
Among the trials for analyses, by HPLC, several difficulties emerged due to the interaction between copper and acetonitryl used in the mobile phase and overlap of peaks.
Selective spectroflurometeric method is needed to be developed to monitor the stability of the preparation, active ingredient and conduct pharmacokinetic studies.

### DISCLOSURE OF INVENTION

Preparation and crystal structure of [CuCl (Nicotinic acid)2 H20]complex

### Solution A:

Dissolve nicotinic acid (123 gm) and ascorbic acid (20 gm) in 90% aqueous ethanol (2 L) by gentle warming.

### Suspension B:

Suspend freshly prepared Cu(I)Cl (33 gm) in absolute ethanol (IL).

### Preparation of Nicotinic/Cu (I) Complex:

With constant and efficient stirring add suspension B to solution A drop wise when reddish brown precipitate is formed. Continue stirring for further five minutes after the complete addition of suspension B
Boil the mixture for 5 minutes and then filter by suction.

### Isolation of the Complex:

Filter the reddish brown precipitate and wash with aqueous ascorbic acid 5% w/v solution then with ethanol 90% and finally with least amount of acetone.
Leave to dry in air over night. Yield: 92 gm.

### Storage:

Keep protected from light and under dry conditions at room temperature.

Analytical Data:

| | | | | |
|---|---|---|---|---|
| Found % | C: 39.76 | H: 3.73 | N: 7.45 | Cu: 17.17 |
| Calc. % for: | C: 39.72 | H: 3.31 | N: 7.72 | Cu: 17.37 |

Molecular weight: 362.5

### Physical Properties:

Color: Bright reddish-brown microcrystalline powder

### Stability:

The complex is sufficiently stable when well dried and protected from light

### Solubility:

The complex is insoluble in non polar solvents, e.g. benzene, carbon tetrachloride, etc., and insoluble in polar solvents: water, methanol, ethanol, acetone, but soluble in these solvents upon heating in inert atmosphere, otherwise oxidation takes place.

Soluble in DMF and DMSO

### Infrared Spectrum:

exhibits C═O band around 1710 cm−1, and C—O around 1310 cm−1(KBr pellets)

The composition of the complex formulated as [CuCl(nicotinic acid)2] was confirmed by the single crystal diffraction.

### X-Ray Crystallography:

### Empirical Formula:

C12H12N2O5ClCu, formula weight: 362.5

Monoclonic, space group Pc (No. 7).

Unit cell dimensions: A=3.7752 (5) at 506, B=6.301(1) & A ring, C=26.754 (8) & A ring Beta=90.96 at 730, (2).

The X-ray single crystal data was collected on Nicollet R3m four circle diffract meter.

Crystal size 0.35×0.24×0.15 mm. Graphite monoachrmoatized Mo K @# 945, Radiation (@# 955=0.71069 &A ring). With the &# 969. Scan technique was used to collect the data set. The accurate unit cell parameters were determined and refined by least-squares methods.

Reversed phase—High performance liquid chromatography (RP-HPLC) revealed a single peak at Rt 3.06 min.
Column: C18
Mobile phase: Methanol Acctonitrate
UV detector: 240 nm
Volume injected: 20 μL
Model of apparatus: Gilson

The structure was solved by direct methods and refined by full matrix least squares package. Goodness of fit on the F2 is 1.258. Maximum and minimum peaks in the final difference Fourier synthesis were 0.049 and 0.34 e@# 0506, −3.

According to one broad form of the invention there is provided a process for the preparation of nicotinic acid-copper chloride complex by dissolving the components in a polar solvent e.g. acetone, water, ethanol, . . . as a red bright microcrystalline powder.

According to another broad form of the invention there is provided a m comprises administering to say human person an effective amount of the complex in a pharmaceutically acceptable composition with other acceptable vitamins, carriers, diluent and or excipient. The concentration of the complex and other components depends on different factors, e.g. type and reason of weakness of the muscles, age of the patient, etc.

According to another broad form of the invention there is provided a method for treatment of Fatigue. The method comprises administering to say a human person the copper chloride-nicotinic acid complex and/or a pharmaceutically acceptable composition of the complex. The concentration of the complex in the pharmaceutically acceptable form is variable and depends on several factors, e.g. age of the patient, reason of fatigue, etc. According to a further broad form of the invention there is provided a method for treatment of infertility in men, The method comprises administering to say a man the nicotinic acid complex or apharmaceutically acceptable composition containing the complex, acceptable carrier, diluent, excipient.

Solid dosage forms for oral administration may include capsules, tablets, pills and granules, in such solid dosage forms, the nicotinic acid-copper chloride complex may be admixed with at least one inert diluent

Such dosage forms may also comprise, as normal practice, additional substances other than inert diluents, e.g. ascorbic acid, other vitamins, etc. In the case of capsules the dosage forms may also comprise buffering agents.

### BRIEF DESCRIPTION OF DRAWINGS

FIG. 1:

The copper Nicotinate complex ratio 2:1 gives 2 peaks with RP-HPLC analysis indicating a heterogeneous compound containing other compound beside the active principle “copper Nicotinate”.

FIG. 2:

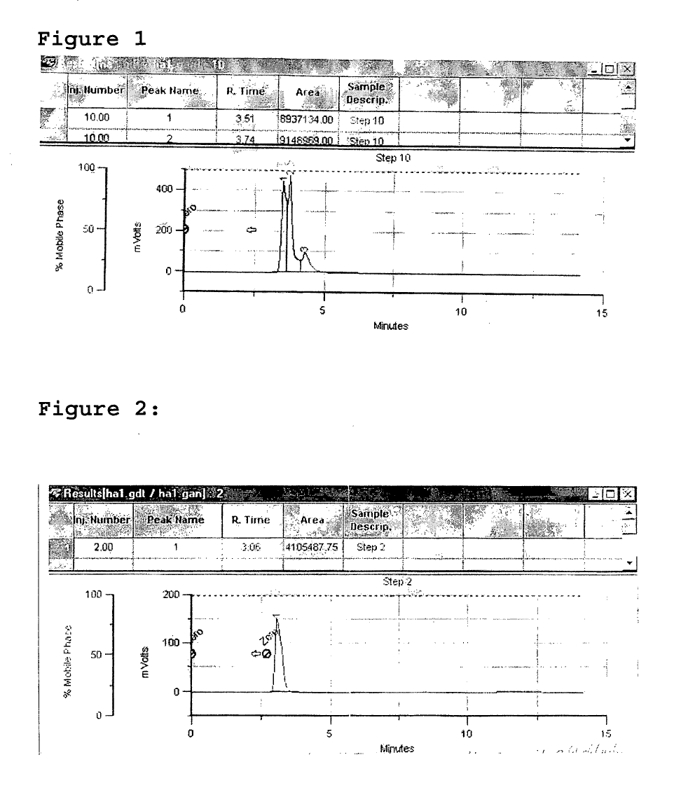

Homogenous copper nicotinic acid complex by RP-HPLC analysis

Reversed phase—High performance liquid chromatography (RP-HPLC) revealed a single peak at Rt 3.06 min.
Column: C18
Mobile phase: Methanol Acetonitrate
UV detector: 240 nm
Volume injected: 20 μL
Model of apparatus: Gilson

### BEST MODE AND OTHER MODES FOR CARRYING OUT THE INVENTION

An effective amount of a copper chloride-nicotinic acid complex, to achieve a desired level of improving fatigue, infertility, weakness of muscles, etc., is administered orally to a human person. The composition for this purpose is presented as capsules, tablets, etc. The specific dose level for a particular person depends on a variety of factors including age, general health, sex, diet, body weight, time of administration, as will be mentioned in details for each case.

1-17. (canceled)

18. The method for preparation of nicotinic acid copper chloride complex, the method for which comprises adding, drop wise, a suspension of copper (I) chloride in aqueous alcohol, to nicotinic acid dissolved in aqueous alcohol to obtain a reactant solution, from which develops a clear reddish precipitate of the nicotinic acid copper (I) chloride complex, having the chemical formula:
C₁₂H₁₂ClCuN₂O₅.

19. In the method according to claim 18 the final mixture was allowed to stand over several hours until the crystalline precipitate was formed.

20. In the method according to claim 19, the precipitate was filtered utilizing a pump and washed with ascorbic acid solution in alcohol, the solution comprising 70-90% ascorbic acid, further washings with ethanol 70-99%, acetone and dried under vacuum.

21. In the method according to claim 18 where the reactant solution is subject to boiling.

22. A nicotinic acid copper (I) chloride complex prepared in accordance with the method of claim 18, having the chemical formula:
C₁₂H₁₂ClCuN₂O₅.

23. A pharmacologically active composition for the treatment of patients suffering from muscular dystrophy, the composition comprising a unit dosage amount of a nicotinic acid copper (I) chloride complex, and a physiologically suitable carrier.

24. The pharmacologically active composition for the treatment of patients suffering from muscular dystrophy according to claim 23, comprising in addition to the physiologically suitable carrier a material selected from the group consisting of vitamins, diluents and excipients.

25. A pharmacologically active composition for the treatment of patients suffering from muscular dystrophy according to claim 23, in a solid dosage form for oral administration selected from the group consisting of capsules, tablets, pills, and granules.

26. A pharmacologically active composition for the treatment of patients suffering from burns or scars, the composition comprising a nicotinic acid copper (I) chloride complex in a semisolid dosage form for topical skin application.

27. A pharmacologically active composition according to claim 23, for the treatment of patients suffering from Parkinsons disease, myasthenia gravis, spermatogenesis, and muscle weakness of different etiologies.

28. A pharmacologically active composition according to claim 23, for the stimulation of stem cells in a patient.

29. A pharmacologically active composition according to claim 23, for inducing mitochondrial regeneration and multiplication at the pathological site of a patient.

30. A pharmacologically active composition according to claim 23, intended to induce the catalytic release of nitric oxide from thionitric oxide (SNO) compounds formed endogenously in a patient.

31. pharmacologically active composition according to claim 23, for the treatment of fatigue.

32. A pharmacologically active composition according to claim 23, in semisolid dosage form for the treatment of male infertility.
